(19) United States
(12) Patent Application Publication
(10) Pub. No.: US 2026/0294397 A1
(43) Pub. Date: Oct. 1, 2026

(54) ULTRASOUND PROBE

(71) Applicant: FUJIFILM Corporation, Tokyo (JP)

(72) Inventors: Gen SHIINA, Tokyo (JP); Nobutaka MIYAMOTO, Tokyo (JP); Hitoshi TAKETOMO, Tokyo (JP); Kento UI, Tokyo (JP); Yudai SATO, Tokyo (JP)

(73) Assignee: FUJIFILM Corporation, Tokyo (JP)

(21) Appl. No.: 19/568,697

(22) Filed: Mar. 16, 2026

(30) Foreign Application Priority Data
Mar. 31, 2025 (JP) 2025-057455

(51) Int. Cl. A61B 8/00

(52) U.S. Cl. CPC A61B 8/4455; A61B 8/4488

(57) ABSTRACT

An ultrasound probe includes: a head part that incorporates a transducer array in which transducers that transmit and receive ultrasonic waves to and from a subject are arranged; and a grip part that has a rod shape and is connected to the head part. Two recesses arranged along a longitudinal direction of the grip part are formed on each of left and right side surfaces of the grip part. A size of a hosel side recess that is one recess on a head part side among the two recesses is smaller than a size of a grip end side recess that is the other recess.

### CROSS REFERENCE TO RELATED APPLICATION

This application claims priority to Japanese Patent Application No. 2025-057455 filed on (March 31, 2025) which is incorporated herein by reference in its entirety including the specification, claims, drawings, and abstract.

### BACKGROUND

### 1. Technical Field

The present disclosure relates to an ultrasound probe, and particularly to a shape of the ultrasound probe.

### 2. Description of the Related Art

An ultrasound probe that transmits and receives ultrasonic waves to and from a subject is known. An ultrasound image such as a tomographic image used for ultrasound diagnosis is formed based on the received ultrasonic waves. The ultrasound probe includes a head part and a grip part. A transducer array is built in the head part. An operator holds the grip part and brings the head part into contact with a diagnosis region of the subject. Then, a position and a posture of the ultrasound probe are changed by an operation such that an ultrasound image suitable for the diagnosis is obtained. Further, the ultrasound probe may be pressed against the subject depending on a situation.

Various shapes of the ultrasound probe have been proposed in consideration of a region of the subject that comes into contact with the head part and the operability of the operator, that is, ease of adjusting the position and the posture of the ultrasound probe. Further, the burden on a body of the operator such as the reduction in fatigue during long-term operation and the reduction in fatigue of fingers is also considered. In particular, the shape of the grip part is required to be suitable for changing the position and the posture of the ultrasound probe, for handling such as holding, pressing, and lifting, and for the grip in accordance with various ways of holding. Various shapes of the grip part have been proposed as described in JP6677049B, JP4616750B, Design Registration No. 1593697, and Design Registration No. 1591725

### SUMMARY

There is room for improvement in the shape of the ultrasound probe, particularly the shape of the grip part.

An aspect of the present disclosure relates to an ultrasound probe comprising: a head part that incorporates a transducer array in which transducers that transmit and receive ultrasonic waves to and from a subject are arranged; and a grip part that has a rod shape and is connected to the head part. Two finger holding parts arranged along a longitudinal direction of the grip part are formed on each of left and right side surfaces of the grip part. Each of the finger holding parts has, on a head part side and on a side opposite to the head part side, respective stop structures on which a finger of an operator is stopped, such that the finger of the operator is received between the stop structures. A size of a hosel side finger holding part that is one finger holding part on the head part side among the two finger holding parts is smaller than a size of a grip end side finger holding part that is the other finger holding part.

In a case of finely operating the ultrasound probe, the operator grips the position of the grip part close to the head part. In this case, the operator can stabilize a gripping position by gripping a portion of a relatively small finger holding part (hosel side finger holding part). In addition, in a case of moving the ultrasound probe to a large extent, the operator can accommodate a gripping position preferred by the operator by disposing a relatively large finger holding part (grip end side finger holding part) at the position gripped by the operator.

In the above-described ultrasound probe, a size of the finger holding part may represents along the longitudinal direction of the grip part, and a length of the hosel side finger holding part is shorter than a length of the grip end side finger holding part.

In the above-described ultrasound probe, the hosel side finger holding part may have a circular shape, and the grip end side finger holding part may have an oval shape that is long in the longitudinal direction of the grip part. With the circular finger holding structure, the finger is less likely to slip in any direction in a case where the finger holding structure is gripped. In addition, with the oval recess, the operator can grip the ultrasound probe at any position in the longitudinal direction of the obal shape depending on the preference.

In the above-described ultrasound probe, dimensions of the hosel side finger holding part and the grip end side finger holding part in a direction orthogonal to the longitudinal direction of the grip part may be equal to each other. In a case where the gripping position is changed between the hosel side finger holding part and the grip end side finger holding part, the same gripping feeling can be maintained.

In the above-described ultrasound probe, the grip part may be connected to a heel part of the head part and is disposed obliquely upward and rearward with respect to an arrangement direction of the transducers, and a step may be formed on a rear side surface of the grip part such that a portion of the grip part on the head part side from a hosel side finger holding part is thinner than a portion of the grip part on the other side from the hosel side finger holding part. Since the grip part is thin on the head part side, the position of the finger that comes into contact with the rear side surface of the grip part is stabilized, and the ultrasound probe can be firmly held.

In the above-described ultrasound probe, a concave portion curved in a front-rear direction may be formed at a portion of a front side surface of the grip part adjacent to the head part. In a case of observing a flexion and extension state of the finger, a fingertip can be prevented from interfering with the grip part.

In the above-described ultrasound probe, the grip part may have planar parts on the left and right side surfaces at a portion in which the two finger holding parts are not formed. The operator who holds the grip part can recognize the orientation of the head part.

In the above-described ultrasound probe, the two finger holding parts may be provided at an interval. A portion between the two recesses serves as a guide in a case of rotating the ultrasound probe around an axis of the grip part.

In the above-described ultrasound probe, the two finger holding parts may be formed by recesses.

Another aspect of the present invention relates to an ultrasound probe comprising: a head part that incorporates a transducer array in which transducers that transmit and receive ultrasonic waves to and from a subject are arranged; and a grip part that is connected to the head part, in which two recesses arranged along a longitudinal direction of the grip part are formed on each of left and right side surfaces of the grip part, and a length of one recess on a head part side among the two recesses in the longitudinal direction of the grip part is shorter than a length of the other recess in the longitudinal direction of the grip part.

By forming the finger holding parts having different dimensions on the side surface of the grip part of the ultrasound probe, the operability in various operations is improved. By making the hosel side finger holding part close to the head part small, the operator can stably hold the ultrasound probe in a case of finely operating the ultrasound probe. In addition, by making the finger holding part away from the head part long, the operator can stably hold the ultrasound probe at various positions.

### BRIEF DESCRIPTION OF THE DRAWINGS

FIG. 1 is a perspective view illustrating an ultrasound probe according to the present embodiment.

FIG. 2 is a side view illustrating the ultrasound probe according to the present embodiment.

FIG. 3 is a view illustrating a state in which the ultrasound probe according to the present embodiment is viewed from above along a longitudinal direction of a grip part.

FIG. 4 is a view illustrating a state in which the ultrasound probe according to the present embodiment is viewed from below along the longitudinal direction of the grip part.

FIG. 5 is a cross-sectional view taken along line A-A in FIG. 2.

FIG. 6 is a cross-sectional view taken along line B-B in FIG. 2.

FIG. 7 is a cross-sectional view taken along line C-C in FIG. 2.

FIG. 8 is a view illustrating a dimension of a recess provided on a side surface of the ultrasound probe.

FIG. 9 is a side view illustrating an ultrasound probe according to another embodiment.

### DESCRIPTION OF EMBODIMENTS

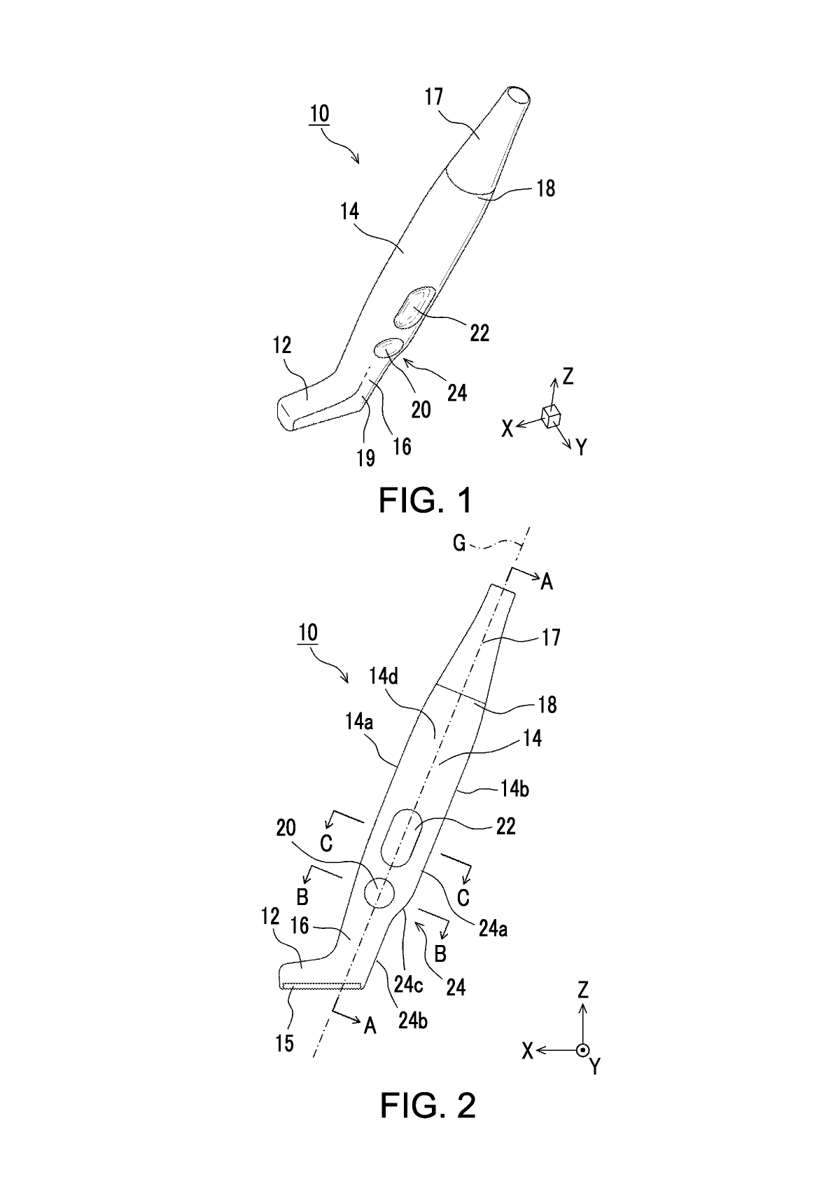

Hereinafter, embodiments of the present invention will be described with reference to the drawings. FIG. 1 is a perspective view illustrating a schematic shape of an ultrasound probe 10 according to the present embodiment. The ultrasound probe 10 includes a head part 12 and a grip part 14. The head part 12 incorporates a transducer array 15 (see FIG. 2) in which ultrasound transducers are arranged. The grip part 14 is a part that is gripped by an operator during the operation, and has a substantially rod shape. An axis of the grip part 14 is referred to as an axis G. The head part 12 is connected to one end of the grip part 14. An end part of the grip part 14 on a side to which the head part 12 is connected is referred to as a hosel 16. The ultrasound probe 10 is connected to an ultrasound diagnostic apparatus (not illustrated) via a cable (not illustrated). A cable boot 17 is connected to an end of the grip part 14 opposite to the hosel 16. The end of the grip part 14 to which the cable boot 17 is connected is referred to as a grip end 18.

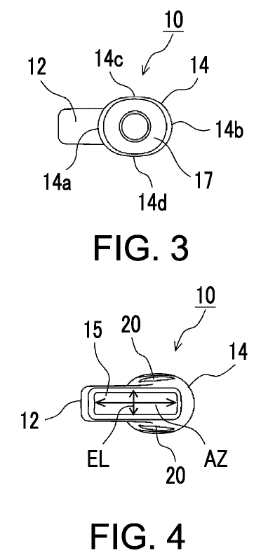

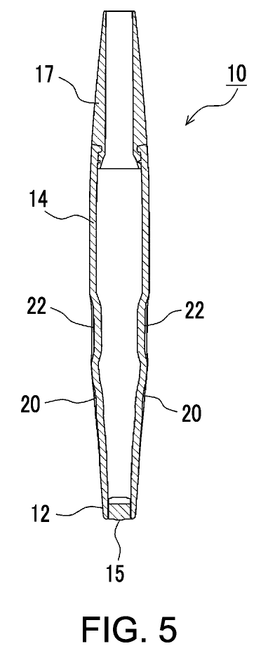

FIG. 2 is a side view illustrating a left side of the ultrasound probe 10. A right side surface is symmetrical to the side surface illustrated in FIG. 2. FIG. 3 is a view of the ultrasound probe 10 as viewed from above along the axis G of the grip part 14 in FIGS. 2, and 4 is a view as viewed from below. FIGS. 5-7 are cross-sectional views taken along line A-A, line B-B, and line C-C in FIG. 2, respectively.

An azimuth direction AZ (see FIG. 4) that is an arrangement direction of a large number of ultrasound transducers constituting the transducer array 15 is a left-right direction in FIG. 2, and a direction along this direction is defined as an X-axis direction of a three-dimensional orthogonal coordinate system. A positive direction (a direction of an arrow X in FIG. 2) of the X-axis is a front direction, and a negative direction thereof is a rear direction. Among directions orthogonal to the azimuth direction (X-axis direction), a direction in a paper plane of FIG. 2 is a Z-axis direction, and a positive direction (a direction of an arrow Z in FIG. 2) of the Z-axis is an up direction, and a negative direction is a down direction. Further, among the directions orthogonal to the azimuth direction (X-axis direction), a direction vertically penetrating the paper plane of FIG. 2 is a Y-axis direction, and a positive direction (a front direction of the paper plane of FIG. 2) of the Y-axis is referred to as a left direction, and a negative direction (a rear direction of the paper plane of FIG. 2) is referred to as a right direction. Further, a direction of the transducer array 15 orthogonal to the azimuth direction AZ is referred to as an elevation direction EL (see FIG. 4). In the following description, unless otherwise noted, the relative positional relationship, the direction, and the orientation follow the above definitions.

The transducer array 15 is disposed on a lower surface of the head part 12. The transducer array 15 may include an acoustic lens, and the acoustic lens may be a portion of a housing that defines an outer shape of the head part 12. The transducer array 15 transmits an ultrasonic beam downward in FIG. 2. The transducer array 15 receives the ultrasonic waves reflected from within the subject.

The head part 12 is substantially cuboid, and an upper surface thereof may be inclined in the X-axis direction such that the head part 12 is slightly narrowed toward a distal end of the head part 12 as illustrated in FIG. 2. The grip part 14 is connected to a rear end part (hereinafter, referred to as a heel part 19) of the head part 12. The grip part 14 is disposed such that the axis G thereof is oblique upward and rearward with respect to the longitudinal direction of the head part 12. The axis G of the grip part 14 may be inclined at 110° to 120° with respect to the X-axis direction, and may be connected to the heel part 19. In addition, the grip part 14 may be connected to the head part 12 such that a rear surface of the hosel 16 of the grip part 14 is coplanar with a rear end surface of the heel part 19 of the head part 12. Since the grip part 14 is disposed to be inclined rearward with respect to the heel part 19 of the head part 12, the ultrasound probe 10 has a shape of a stick used in a hockey game as a whole.

As illustrated in FIG. 3, the grip part 14 has a cross-sectional shape orthogonal to the axis G that is long in the X-axis direction, for example, a substantially oval shape. More specifically, the cross-sectional shape of the grip part 14 is substantially a semicircle having substantially the same diameter on the front and rear sides, and the left and right sides may be formed by straight lines connecting ends of the substantially semicircular front and rear sides. Therefore, the grip part 14 has a curved front side surface 14a and a curved rear side surface 14b, and has planar parts facing the right side and the left side, respectively, on a right side surface 14c and a left side surface 14d.

Two recesses 20 and 22 are formed on each of the left and right side surfaces 14d and 14c of the grip part 14. The two recesses 20 and 22 are disposed side by side along the longitudinal direction of the grip part 14. The recess 20 on the hosel 16 side is referred to as a hosel side recess 20, and the recess 22 on the grip end 18 side is referred to as a grip end side recess 22. In the longitudinal direction of the grip part 14, a length of the hosel side recess 20 is shorter than a length of the grip end side recess 22. The hosel side recess 20 and the grip end side recess 22 are disposed to be spaced from each other, and a portion of the grip part 14 between the two recesses 20 and 22 has a substantially oval cross-sectional shape that is long in the X-axis direction.

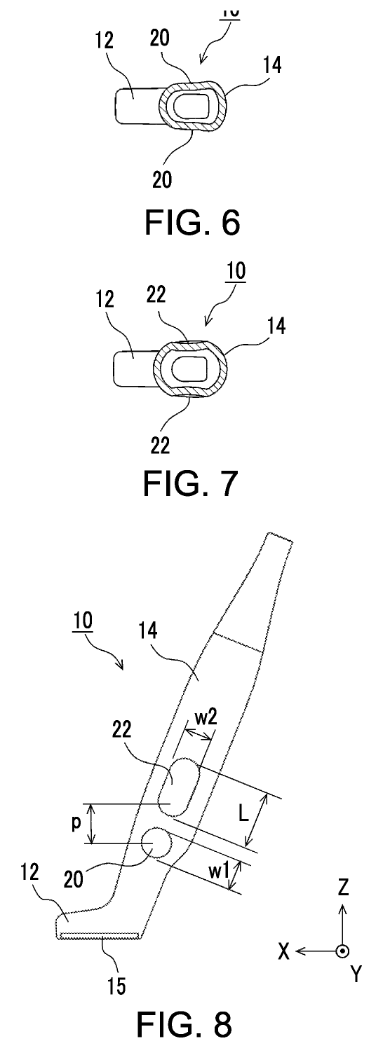

A shape of the hosel side recess 20 may be substantially circular. As illustrated in cross-sectional views of FIGS. 5 and 6, the hosel side recess 20 is recessed such that a peripheral edge is high and a center is low over the entire circumference. A bottom surface of the hosel side recess 20 may have a shape that is gradually deeper as it approaches the center from the peripheral edge, and may be, for example, a portion of a spherical surface. A diameter w1 (see FIG. 8) of the hosel side recess 20 may be 8 to 18 mm, and a depth thereof may be 0.3 to 0.4 mm.

The grip end side recess 22 may have a substantially oval shape that is long in the longitudinal direction of the grip part 14. Specifically, a shape of a contour of the grip end side recess 22 is a shape consisting of substantially semicircular arcs facing each other and two substantially parallel straight lines connecting ends of the substantially semicircular arcs. As illustrated in FIGS. 5 and 7, the grip end side recess 22 is recessed such that a peripheral edge is high and the height is gradually lowered as it approaches the center over the entire circumference. A substantially semicircular portion of the bottom surface of the grip end side recess 22, which is defined by substantially semicircular arcs at both ends of the substantially oval shape and a diameter connecting both ends of each substantially semicircular arc, may be a portion of a spherical surface. A portion connecting the substantially semicircular portions may have a groove shape extending along the longitudinal direction of the grip part 14. A bottom surface of the groove may have a shape that is deeper as it approaches a center line in a groove width direction, and may be, for example, a portion of a cylindrical surface. A length L (see FIG. 8) of the grip end side recess 22 in the longitudinal direction of the grip part 14 may be 16 to 60 mm, and a dimension orthogonal to the longitudinal direction, that is, a width w2 (see FIG. 8) may be 8 to 18 mm. The width w2 of the grip end side recess 22 may be the same dimension as the diameter of the hosel side recess 20. A depth of the grip end side recess 22 may be 0.3 to 0.4 mm, and may be the same dimension as the depth of the hosel side recess 20.

An arrangement interval p between the hosel side recess 20 and the grip end side recess 22 in the Z-axis direction may be 10 to 35 mm. The arrangement interval p is an interval in the Z-axis direction between the center of the hosel side recess 20 and the center of a substantially semicircular portion on the hosel side of the grip end side recess 22. The arrangement interval p may be determined to be a dimension in which a space is present between the hosel side recess 20 and the grip end side recess 22.

A step is provided on the rear side surface 14b of the grip part 14. This step is referred to as a rear surface step 24. The rear surface step 24 consists of a high surface 24a, a low surface 24b, and a transition surface 24c connecting the surfaces. The high surface 24a and the low surface 24b may be substantially parallel surfaces, and the transition surface 24c may be inclined relative to the high surface 24a and the low surface 24b. The transition surface 24c is positioned at substantially the same position as the hosel side recess 20 in the longitudinal direction of the grip part 14. The high surface 24a is positioned on the grip end side from the position of the hosel side recess 20 in the longitudinal direction of the grip part 14, and the low surface 24b is positioned on the hosel side from the position of the hosel side recess 20. The rear surface step 24 causes the grip part 14 to be thinner on the hosel side than the hosel side recess 20 and to be thicker on the grip end side than the hosel side recess 20.

In a case where the operator places the finger on the hosel side recess 20 to operate the ultrasound probe 10, the hosel side recess 20 is a finger holding part that holds the finger at the position. In a case where the operator further presses the ultrasound probe 10 against the diagnosis region of the subject, the finger is stopped on an inclined surface of the hosel side recess 20 on the hosel 16 side, and the finger is held in the hosel side recess 20. This inclined surface on the hosel 16 side is a stop structure on which the finger of the operator is stopped. Since the finger of the operator is stopped on the inclined surface of the hosel side recess 20, the finger is less likely to slip, and a force of pressing the ultrasound probe 10 against the diagnosis region can be reliably transmitted. On the contrary, in a case where the operator operates the ultrasound probe 10 to separate the ultrasound probe 10 from the diagnosis region of the subject, the finger is stopped on an inclined surface of the hosel side recess 20 on the grip end 18 side, and the finger is held in the hosel side recess 20. This inclined surface on the grip end 18 side is a stop structure on which the finger of the operator is stopped. Since the finger of the operator is stopped on the inclined surface on the grip end side, the finger is less likely to slip in a case of separating the ultrasound probe 10 from the diagnosis region. The finger of the operator is held between the inclined surface on the hosel 16 side and the inclined surface on the grip end 18 side.

In the grip end side recess 22, the inclined surface on the hosel 16 side and the inclined surface on the grip end 18 side are stop structures on which the finger of the operator is stopped, as in the hosel side recess 20, and the finger is held between these stop structures. The grip end side recess 22 is a finger holding part that holds the finger in contact with the grip end side recess 22. In the grip end side recess 22, as in the hosel side recess 20, the finger is less likely to slip in a case of pressing the ultrasound probe 10 against the diagnosis region and in a case of separating the ultrasound probe 10 from the diagnosis region.

In an operation of moving the ultrasound probe 10 along a surface of the diagnosis region in a state in which the ultrasound probe 10 is in contact with the diagnosis region of the subject, or changing a posture to adjust an angle with respect to the surface of the diagnosis region, the hosel side recess 20 and the grip end side recess 22 improve the operability. In such an operation, the operator holds the grip part 14 by pinching the grip part 14 with fingers from the left and right, and places the finger on at least one of the hosel side recess 20 or the grip end side recess 22. Since the pad of the finger is in contact with the recess, the contact area is increased, and the ultrasound probe 10 can be held with a light force. Since the size of the hosel side recess 20 is small, the entire pad of the finger is in contact with the hosel side recess 20, and the position and the posture of the ultrasound probe 10 can be easily fine-tuned. On the other hand, since the size of the grip end side recess 22 is large, the degree of freedom of the position at which the finger is placed is high, and various grips are possible.

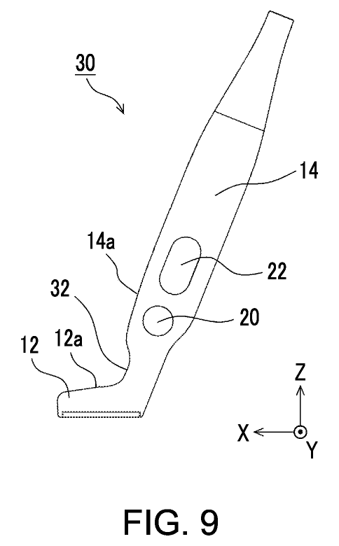

FIG. 9 is a side view illustrating an outer shape of an ultrasound probe 30 according to another embodiment. In the ultrasound probe 30, a concave portion 32 is formed in a region of the front side surface 14a of the grip part 14 on the hosel side. The ultrasound probe 30 has the same configuration as the ultrasound probe 10 described above, except that the concave portion 32 is formed. The concave portion 32 is provided at a corner portion formed by the upper surface 12a of the head part 12 and the front side surface 14a of the grip part 14. In addition, the concave portion 32 may be formed rearward. The upper surface 12a of the head part 12 extends straight in the region of the concave portion 32, and a length of the upper surface 12a of the head part 12 in this case is longer than a length in a case where the concave portion 32 is not provided. For example, in a case where the subject's finger is diagnosed and the lower surface of the head part 12 is brought into contact with the pulp side and the position of the proximal phalanx of the finger of the subject and the state of flexion and extension of the finger of the subject is observed, the finger joint of the subject is bent such that the finger of the subject is wound around the distal end of the head part 12 while the head part 12 is brought into contact with the pulp side and the position of the proximal phalanx of the finger of the subject. In this case, the presence of the concave portion 32 prevents the fingertip of the subject from interfering with the grip part 14.

The finger holding part provided on the grip part 14 is not limited to a shape recessed from a general surface of the side surface of the grip part 14 as in the hosel side recess 20 or the grip end side recess 22 described above. For example, the finger holding part may be an annular ridge that protrudes from the side surface of the grip part 14. The ridge may be substantially circular or substantially oval, as in the peripheral edge shape of the hosel side recess 20 or the grip end side recess 22. A top of this ridge may be rounded.

The shape of the finger holding part is not limited to a circular or oval shape, and may be another rounded shape. For example, the shape of the finger holding part may be substantially elliptical or substantially quadrangular with rounded corners (substantially rounded quadrangular). Each side of the substantially rounded quadrangular shape may be a curve having a curvature radius larger than a curvature radius of a corner, and a curve of the corner and a curve of the side may be smoothly connected.

The shape of the grip part 14 may be a medium-thick rod shape that is thick at a center portion and thin at end parts in the longitudinal direction. In addition, a shape of a cross section of the grip part 14 orthogonal to the axis G may be substantially oval, substantially elliptical, or substantially rectangular with rounded corners that is long in the X-axis direction. Since the left and right side surfaces 14d and 14c of the grip part 14 are planar or slightly curved surfaces and boundaries between adjacent side surfaces of the four side surfaces of the grip part 14 are rounded, the ultrasound probes 10 and 30 can be used for a long time without causing a feeling of fatigue, which helps to maintain reliable operation.

What is claimed is:

1. An ultrasound probe comprising:
a head part that incorporates a transducer array in which transducers that transmit and receive ultrasonic waves to and from a subject are arranged; and
a grip part that has a rod shape and is connected to the head part,
wherein two finger holding parts arranged along a longitudinal direction of the grip part are formed on each of left and right side surfaces of the grip part, each of the finger holding parts having, on a head part side and on a side opposite to the head part side, respective stop structures on which a finger of an operator is stopped, such that the finger of the operator is received between the stop structures, and
a size of a hosel side finger holding part that is one finger holding part on the head part side among the two finger holding parts is smaller than a size of a grip end side finger holding part that is the other finger holding part.

2. The ultrasound probe according to claim 1,
wherein a size of the finger holding part represents along the longitudinal direction of the grip part, and a length of the hosel side finger holding part is shorter than a length of the grip end side finger holding part.

3. The ultrasound probe according to claim 1,
wherein the hosel side finger holding part has a circular shape, and the grip end side finger holding part has an oval shape that is long in the longitudinal direction of the grip part.

4. The ultrasound probe according to claim 3,
wherein dimensions of the hosel side finger holding part and the grip end side finger holding part in a direction orthogonal to the longitudinal direction of the grip part are equal to each other.

5. The ultrasound probe according to claim 1,
wherein the grip part is connected to a heel part of the head part and is disposed obliquely upward and rearward with respect to an arrangement direction of the transducers, and
a step is formed on a rear side surface of the grip part such that a portion of the grip part on the head part side from a hosel side finger holding part is thinner than a portion of the grip part on the other side from the hosel side finger holding part.

6. The ultrasound probe according to claim 5,
wherein a concave portion curved in a front-rear direction is formed at a portion of a front side surface of the grip part adjacent to the head part.

7. The ultrasound probe according to claim 1,
wherein the grip part has planar parts on the left and right side surfaces at a portion in which the two finger holding parts are not formed.

8. The ultrasound probe according to claim 7,
wherein the two finger holding parts are provided at an interval.

9. The ultrasound probe according to claim 1,
wherein the two finger holding parts are formed by recesses.

10. An ultrasound probe comprising:
a head part that incorporates a transducer array in which transducers that transmit and receive ultrasonic waves to and from a subject are arranged; and
a grip part that is connected to the head part,
wherein two recesses arranged along a longitudinal direction of the grip part are formed on each of left and right side surfaces of the grip part, and a length of one recess on a head part side among the two recesses in the longitudinal direction of the grip part is shorter than a length of the other recess in the longitudinal direction of the grip part.
